(19) United States
(12) Patent Application Publication
(10) Pub. No.: US 2015/0164482 A1
(43) Pub. Date: Jun. 18, 2015

(54) ULTRASOUND DIAGNOSTIC APPARATUS, ULTRASOUND IMAGE RECORDING METHOD, AND NON-TRANSITORY COMPUTER READABLE RECORDING MEDIUM

(71) Applicant: KONICA MINOLTA, INC., Tokyo (JP)

(72) Inventor: Bumpei TOJI, Hashima-shi (JP)

(73) Assignee: KONICA MINOLTA, INC., Tokyo (JP)

(21) Appl. No.: 14/567,933

(22) Filed: Dec. 11, 2014

(30) Foreign Application Priority Data
Dec. 12, 2013 (JP) 2013-257209
Dec. 2, 2014 (JP) 2014-244200

(51) Int. Cl. A61B 8/08; A61B 8/00; A61B 8/14

(52) U.S. Cl. CPC A61B 8/5276; A61B 8/14; A61B 8/5207; A61B 8/4263; A61B 8/461

(57) ABSTRACT

An ultrasound diagnostic apparatus connectable to a probe and recording an ultrasound image of the inside of a subject's body based on reflected ultrasound from inside the subject's body acquired by the probe positioned to face a surface of the subject's body, the ultrasound diagnostic apparatus including image recording circuitry including: an ultrasound image generator generating the ultrasound images based on the reflected ultrasound; a probe movement determiner determining, based on motion information of each of the plurality of ultrasound images obtained by analyzing the plurality of ultrasound images, movement of the probe occurring at generation of each of the ultrasound images; a recording image selector selecting at least one ultrasound image from among the ultrasound images according to the determination performed by the probe movement determiner; and an ultrasound image recorder recording therein the ultrasound image selected by the recording image selector.

### CROSS-REFERENCE TO RELATED APPLICATION

This application is based on applications No. 2013-257209 and No. 2014-244200 filed in Japan, the contents of which are hereby incorporated by reference.

### BACKGROUND OF THE INVENTION

(1) Field of the Invention

The present disclosure relates to ultrasound diagnostic apparatuses and diagnostic image recording methods. In particular, the present disclosure relates to an ultrasound diagnostic apparatus and a diagnostic image recording method for specifying a diagnostic image to be recorded, based on reflected ultrasound from a subject, acquired by an ultrasound probe.

(2) Description of the Related Art

An ultrasound diagnostic apparatus transmits ultrasound to the inside of the subject via an ultrasound probe, and receives reflected ultrasound generated due to a difference of acoustic impedance between tissues of the subject. Furthermore, using the reception signal, the ultrasound diagnostic apparatus generates ultrasound cross-sectional images (hereinafter, “ultrasound images”) showing the structure of the inner tissues of the subject, and displays the images on a monitor display (hereinafter, “display device”). Ultrasound diagnostic apparatuses are less-invasive, and are cable of displaying the state of the inner tissues in real time in the form of ultrasound images. Therefore, ultrasound diagnostic apparatuses are commonly used for shape diagnostics of living organisms.

Ultrasound diagnostic apparatuses in recent years can produce high quality ultrasound images with high resolution due to digitization of the apparatuses and their ability to use a higher frequency. In particular, the quality of imaging superficial tissues has been dramatically improved. Consequently, it is now common to use an ultrasound diagnostic apparatus in the field of orthopedics as well, in which evaluation of superficial tissues is important. In the field of orthopedics, examination and treatment is performed with respect to disease of various body parts relating to physical movements (e.g. the limbs and the spine), except for the brains and internal organs. Accordingly, various body parts can be the targets of ultrasound examination.

In ultrasound examinations using a conventional ultrasound diagnostic apparatus, usually the examiner holds an ultrasound probe (hereinafter, “probe”) in one hand, and operates the ultrasound diagnostic apparatus with the other hand. For example, the examiner holds the probe in the right hand, and observes the ultrasound images displayed on a monitor device while moving the probe along the body surface of the subject. Meanwhile, the examiner operates the buttons of the ultrasound diagnostic apparatus with the left hand, in order to input an instruction such as a freeze instruction for recording a desired ultrasound image. Generally, the examiner needs to manipulate the probe at a distance from the ultrasound diagnostic apparatus, and therefore needs to take an unnatural posture in some cases.

Furthermore, it is common in an orthopedic examination that the examiner supports a leg or arm of the subject by one hand during the examination.

As both hands are full, the examiner sometimes has difficulties in operating the ultrasound diagnostic apparatus.

In response to this problem, Japanese Patent Application Publication No. H6-78920 proposes an ultrasound diagnostic apparatus that has a foot switch for inputting a freeze instruction. Also, Japanese Patent Application Publication No. 2002-345812 proposes a probe having a switch, by which the examiner can input a freeze instruction while holding the probe.

### SUMMARY OF THE INVENTION

### Problems to be Solved by the Invention

However, there is a problem that the configurations disclosed in the aforementioned literatures require an additional component such as the foot switch or the switch on the probe.

The present disclosure is made in view of the above-described problem, and aims to provide an ultrasound diagnostic apparatus and an image recording method that allow for an easier freeze operation for selecting and recording a desired ultrasound image.

### Means for Solving the Problems

The above-described aim is achieved by an ultrasound diagnostic apparatus to which a probe is connectable and that records an ultrasound image of the inside of a subject's body based on reflected ultrasound from inside the subject's body acquired by the probe positioned to face a surface of the subject's body, the ultrasound diagnostic apparatus comprising image recording circuitry that includes:

an ultrasound image generator that generates a plurality of ultrasound images of the inside of the subject's body based on the reflected ultrasound; a probe movement determiner that determines, based on motion information obtained by analyzing the plurality of ultrasound images, movement of the probe occurring at generation of each of the plurality of ultrasound images,; a recording image selector that selects at least one ultrasound image from among the plurality of ultrasound images according to the determination performed by the probe movement determiner; and an ultrasound image recorder that records therein said at least one ultrasound image selected by the recording image selector.

### BRIEF DESCRIPTION OF THE DRAWINGS

These and the other objects, advantages and features of the invention will become apparent from the following description thereof taken in conjunction with the accompanying drawings which illustrate a specific embodiment of the invention.

In the drawings:

FIG. 1 illustrates an external view of an ultrasound diagnostic system 100 in which an ultrasound diagnostic apparatus 150 is included;

FIG. 2 is a block diagram illustrating configuration of the ultrasound diagnostic system 100 using the ultrasound diagnostic apparatus 150 pertaining to Embodiment 1;

FIG. 3 is a block diagram illustrating configuration of image recording circuitry 151 of the ultrasound diagnostic apparatus 150;

FIG. 4 is a diagram illustrating motion information extraction from each ultrasound image, performed by an image analyzer 105 of the ultrasound diagnostic apparatus 150;

FIG. 5 is an example of a motion direction histogram used by a probe movement determiner 106 of the ultrasound diagnostic apparatus 150 when determining the movement of the probe, which is either stationary or moving;

FIG. 6 is a flowchart illustrating operation of the ultrasound diagnostic apparatus 150 when outputting an image to be recorded;

FIG. 7 is a flowchart showing details of Steps S202, S203 and S204 of the flowchart shown in FIG. 6.

FIG. 8 is a diagram illustrating motion information of each image area when the image analyzer 105 determines that the probe was stationary, overlaying arrow images X on an ultrasound image;

FIG. 9 is an example of a motion direction histogram showing motion directions detected from the ultrasound image shown in FIG. 8;

FIG. 10 is a diagram illustrating motion information of each image area when the image analyzer 105 determines that the probe was moving, overlaying arrow images X on an ultrasound image;

FIG. 11 is an example of a motion direction histogram showing motion directions detected from the ultrasound image shown in FIG. 10;

FIG. 12 is a flowchart showing details of operation pertaining to Modification 1, in which Steps S202, S203 and S204 of the flowchart shown in FIG. 6 are modified;

FIG. 13 is a diagram illustrating motion information of each image area when the image analyzer 105 determines that inner tissues of the subject's body are moving, overlaying arrow images X on an ultrasound image;

FIG. 14 is an example of a motion direction histogram used in the probe movement determination (Step S203) of the ultrasound diagnostic apparatus 150, illustrating the case where the inner tissues of the subject's body were in the moving state;

FIG. 15 is a flowchart showing details of operation pertaining to a modification in which Steps S202, S203 and S204 of the flowchart shown in FIG. 6 are modified;

FIG. 16 is a block diagram showing configuration of an ultrasound diagnostic system in which an ultrasound diagnostic apparatus 152 pertaining to Embodiment 2 is included;

FIG. 17 is a schematic diagram illustrating images provided by a probe movement state indicator and a recording timing indicator of the ultrasound diagnostic apparatus pertaining to Embodiment 2; and

FIG. 18 is a schematic diagram illustrating an image displayed by the recording timing indicator of the ultrasound diagnostic apparatus pertaining to Embodiment 2.

### DESCRIPTION OF THE PREFERRED EMBODIMENT

The following describes modifications of Embodiment 1.

### Overview of Embodiments

One aspect of the present disclosure is an ultrasound diagnostic apparatus to which a probe is connectable and that records an ultrasound image of the inside of a subject's body based on reflected ultrasound from inside the subject's body acquired by the probe positioned to face a surface of the subject's body, the ultrasound diagnostic apparatus comprising image recording circuitry that includes:

an ultrasound image generator that generates a plurality of ultrasound images of the inside of the subject's body based on the reflected ultrasound; a probe movement determiner that determines, based on motion information obtained by analyzing the plurality of ultrasound images, movement of the probe occurring at generation of each of the plurality of ultrasound images; a recording image selector that selects at least one ultrasound image from among the plurality of ultrasound images according to the determination performed by the probe movement determiner; and an ultrasound image recorder that records therein said at least one ultrasound image selected by the recording image selector.

In response to a determination by the probe movement determiner that the probe was stationary at generation of a given ultrasound image from among the plurality of ultrasound images, the recording image selector may select the given ultrasound image.

In response to a determination by the probe movement determiner that the probe was moving along the surface of the subject's body at generation of every one of the plurality of ultrasound images, the recording image selector may select, from among the plurality of ultrasound images, an ultrasound image showing that the amount of movement of the probe was the smallest.

In response to a determination by the probe movement determiner that the probe was moving away from the surface of the subject's body at generation of a given ultrasound image from among the plurality of ultrasound images, the recording image selector may select, from among the plurality of ultrasound images, an ultrasound image that was generated within a predetermined period that ends at the time the probe was moved away from the surface of the subject's body.

The given ultrasound image may show that the probe was stationary at the generation of the given ultrasound image.

The probe movement determiner may distinguish movement of the probe from movement of tissues inside the subject's body, based on changes in each of the ultrasound images, the changes being indicated by the motion information of each of the ultrasound images.

The image recording circuitry may further include a probe movement state indicator that indicates a probe movement state that is a result of determination performed by the probe movement determiner.

The image recording circuitry may further include a recording timing indicator that indicates a recording timing of an ultrasound image selected by the recording image selector from among the plurality of ultrasound images.

The ultrasound diagnostic apparatus may be connectable to a display device, and may cause the display device to display an ultrasound image selected by the recording image selector from among the plurality of ultrasound images.

The motion information may indicate a motion amount and a motion direction of each of a plurality of image areas of each of the plurality of ultrasound images.

With respect to a given ultrasound image from among the plurality of ultrasound images, when the number of image areas whose motion amount is no greater than a reference value is no less than a predefined threshold value, the probe movement determiner may determine that the probe was stationary at generation of the given ultrasound image, and when the number of the image areas is less than the predefined threshold value, the probe movement determiner may determine that the probe was moving at the generation of the given ultrasound image.

the motion direction detected from each of the plurality of image areas belongs to one of a plurality of motion angle ranges, and

With respect to a given ultrasound image from among the plurality of ultrasound images, when the number of image areas whose motion amount is no greater than a reference value is less than a first threshold value, the probe movement determiner may determine that the probe was stationary at the generation of the given ultrasound image when the number of image areas whose motion amount is greater than the reference value is less than a second threshold value with respect to every one of the plurality of motion angle ranges, the second threshold value being smaller than the first threshold value, and the probe movement determiner determines that the probe was moving at the generation of the given ultrasound image when the number of the image areas whose motion amount is greater than the reference value is no less than the second threshold value with respect to any one of the plurality of motion angle ranges.

The motion angle detected from each of the plurality of image areas may belong to one of a plurality of motion angle ranges, and the probe movement determiner may determine that the probe was stationary at generation of a given ultrasound image when the motion direction of each of the plurality of image areas of the given ultrasound image does not belong to a same one of the plurality of motion angle ranges.

Another aspect of the present disclosure is an ultrasound image recording method for recording an ultrasound image of the inside of a subject's body based on reflected ultrasound from inside the subject's body acquired by a probe positioned to face a surface of the subject's body, comprising: generating a plurality of ultrasound images of the inside of the subject's body based on the reflected ultrasound; determining, based on motion information obtained by analyzing the plurality of ultrasound images, movement of the probe occurring at generation of each of the plurality of ultrasound images; selecting at least one ultrasound image from among the plurality of ultrasound images according to the determining; and recording said at least one ultrasound image.

Another aspect of the present disclosure is a non-transitory computer readable recording medium on which is recorded a program for causing a computer to perform the above-described ultrasound image recording method.

### Embodiment 1

The following explains, with reference to the drawings, an ultrasound diagnostic system 100 in which an ultrasound diagnostic apparatus 150 pertaining to Embodiment 1 is included.

<Overall Configuration of Ultrasound Diagnostic System>

1. Ultrasound Diagnostic Apparatus 150

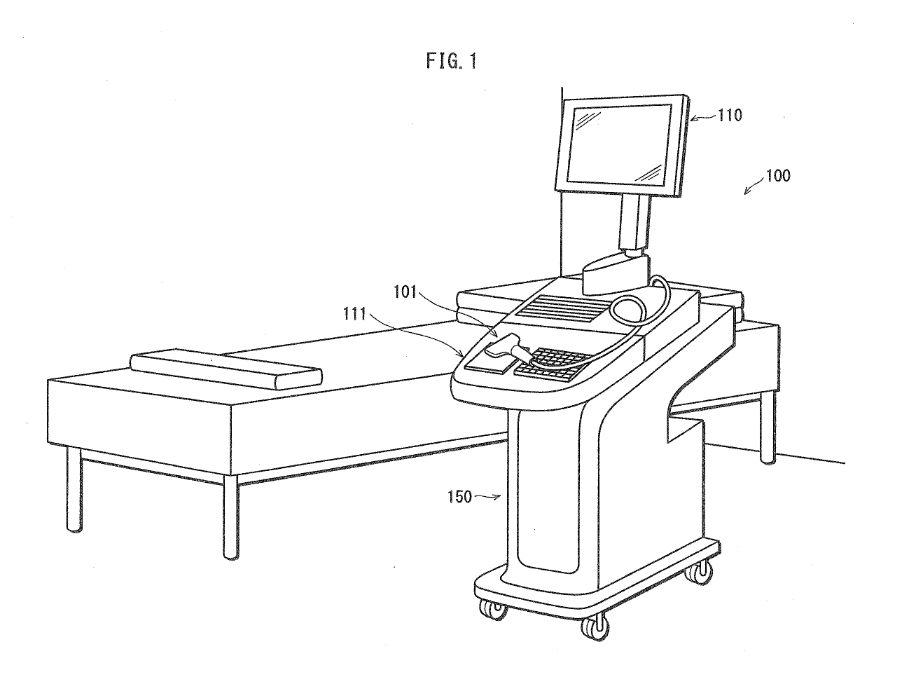

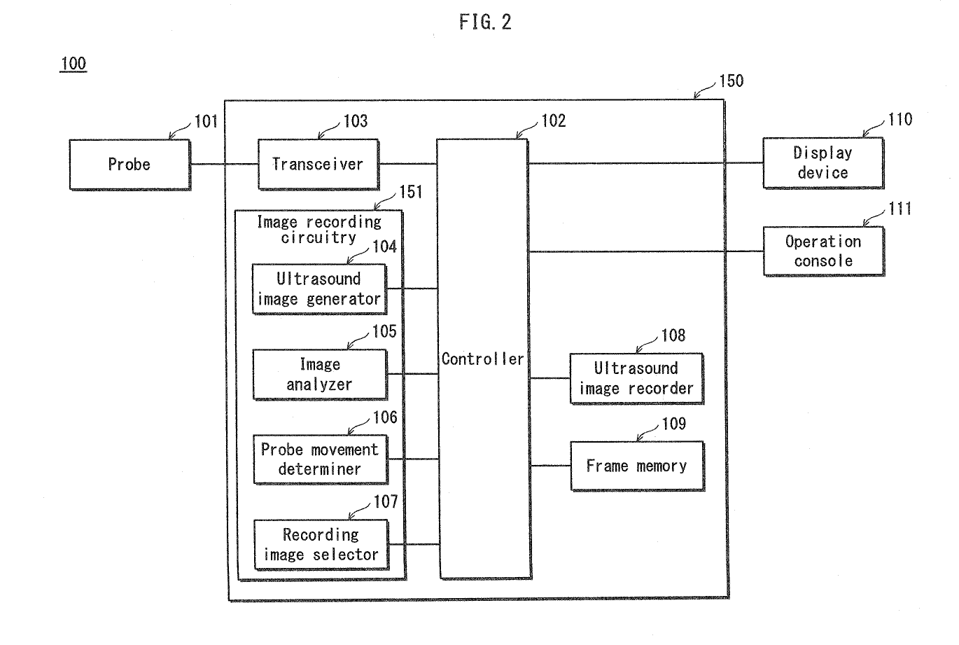

Operations of an ultrasound diagnostic system 100 including an ultrasound diagnostic apparatus 150 pertaining to Embodiment 1 are described below. The ultrasound diagnostic system 100 transmits and receives ultrasound signals for ultrasound diagnosis via a probe 101, and forms images from reflected ultrasound signals so received. FIG. 1 illustrates an external view of the ultrasound diagnostic system 100 in which the ultrasound diagnostic apparatus 150 is included. FIG. 2 is a block diagram illustrating configuration of the ultrasound diagnostic apparatus 150.

As shown in FIG. 1 and FIG. 2, the ultrasound diagnostic system 100 includes the ultrasound diagnostic apparatus 150, the probe 101, a display device 110, and an operation console 111.

The following explains each component included in the ultrasound diagnostic system 100.

As shown in FIG. 2, the ultrasound diagnostic apparatus 150 includes a controller 102, a transceiver 103, an ultrasound image generator 104, an image analyzer 105, a probe movement determiner 106, a recording image selector 107, an ultrasound image recorder 108, and a frame memory 109. Among these components, the ultrasound image generator 104, the image analyzer 105, the probe movement determiner 106 and the recording image selector 107 constitute image recording circuitry 151 of the ultrasound diagnostic apparatus 150.

Each of the elements constituting the ultrasound diagnostic apparatus 150, such as the controller 102, the transceiver 103, the ultrasound image generator 104, the image analyzer 105, the probe movement determiner 106, the recording image selector 107, the ultrasound image recorder 108 and the frame memory 109, is implemented as a hardware circuit, such as field programmable gate array (FPGA) or an application specific integrated circuit (ASIC). Alternatively, each element may be implemented as a combination of programmable device, such as a central processing unit (CPU) or a processor, with software. Each of the aforementioned configuration elements may be single circuit component or may be a collection of circuit components. Further alternatively, a plurality of the aforementioned configuration elements may be combined as a single circuit component or as a collection of circuit components.

The probe 101, the display device 110 and the operation console 111 are provided outside the ultrasound diagnostic apparatus 150, to each of which the ultrasound diagnostic apparatus 150 is connectable. FIG. 2 shows that the probe 101, the display device 110 and the operation console 111 are connected to the ultrasound diagnostic apparatus 150. Note that the probe 101 and the display device 110 may be included in the ultrasound diagnostic apparatus 150.

The following explains configuration of each of the elements connected externally to the ultrasound diagnostic apparatus 150.

2. Probe 101

The probe 101 is an ultrasound probe having ultrasound transducers that transmit and receive ultrasound. The probe 101 has a plurality of transducers (not illustrated) arranged one-dimensionally, for example. The probe 101 converts an electrical signal pulse from the transceiver 103, which is described below, to an ultrasound pulse. The probe 101 emits a plurality of ultrasound beams from the plurality of transducers towards the subject, with the outer surfaces of the transducers of the probe 101 being in contact with the skin surface of the subject. The probe 101 receives a plurality of reflected ultrasound waves (hereinafter, “reflected ultrasound signals”) as echo signals from the subject, converts the reflected ultrasound signals to electrical signals (hereinafter, “reception signals”) by using the plurality of transducers, and provides the reception signals to the transceiver 103.

3. Display Device 110

The display device 110 is a device that displays images. The display device 110 displays, on a display screen, ultrasound images, probe movement determination information, images to be recorded, and so on, which are output via the controller 102 described below. The display device 110 is implemented as a liquid crystal display (LCD) or an organic EL display, for example.

4. Operation Console 111

The operation console 111 is an input device that receives operational input of various settings and operations from an operator with respect to the ultrasound diagnostic apparatus 150 and outputs the operational input to a controller 102. The operator inputs information relating to, for example, a patient name, an examination date, operation or suspension of a screen, storage, and image quality adjustment. Such input information is stored in the ultrasound image recorder 108 or the frame memory 109.

More specifically, the operation console 111 may for example be a keyboard, a trackball, or a touch panel. If the operation console 111 is a touch panel, the operation console 111 may be integrated with the display device 110. In such a configuration, the ultrasound diagnostic apparatus 150 can be operated using the touch panel by performing an operation, such as a touch operation or a drag operation, with respect to an operation key displayed on the display device 110, in order to perform a setting or an operation with respect to the ultrasound diagnostic apparatus 150.

The operation console 111 may alternatively be a keyboard that has keys for performing various operations, or may be an operation panel that has buttons, levers, or the like for performing various operations. Further alternatively, the operation console 111 may be a trackball, a mouse, or any other equipment for moving a cursor displayed on the display device 110. Note that the operation console 111 may alternatively be a plurality of any of the aforementioned types of equipment, or may be a combination of different types of the aforementioned equipment.

<Configuration of Components of Ultrasound Diagnostic Apparatus 150>

The following explains configuration of each of the components included in the ultrasound diagnostic apparatus 150.

1. Controller 102

The controller 102 controls each processing element included in the ultrasound diagnostic apparatus 150. Hereinafter, although no special description is given, the control unit 102 controls operations of the processing units. For example, the control unit 102 causes the processing units to perform processing while controlling an operation timing and so on.

2. Transceiver 103

The transceiver 103 is connected to the probe 101. The transceiver 103 is circuitry that performs transmission processing according to transmission control signals from the controller 102 in order to supply the probe 101 with a transmission ultrasound signal pulse, which enables the probe 101 to transmit an ultrasound beam. Specifically, the transceiver 103 includes a clock generation circuit, a pulse generation circuit, and delay circuit, for example. The clock generation circuit is a circuit that generates a clock signal used for determining transmission timing of the ultrasound beam. The pulse generation circuit is a circuit that generates a pulse signal for driving each of the transducers. The delay circuit is a circuit for performing ultrasound beamforming by setting a delay time for ultrasound beam transmission with respect to each of the transducers and delaying ultrasound beam transmission from each of the transducers by the delay time set with respect thereto.

The transceiver 103 also generates acoustic signal along the depth direction by amplifying the reception ultrasound signal acquired from the ultrasound probe 101 and AD converting the signal to an RF signal, and performing delay-and-sum on the RF signal. The transceiver 103 also performs a reception process of outputting an acoustic signal to the ultrasound image generator 104 in the order of ultrasound scanning.

The RF signal is for example formed from a plurality of signals in the transducer arrangement direction and in an ultrasound transmission direction perpendicular thereto, wherein each of the signals is an amplitude converted electrical signal of reflected ultrasound that is A/D converted to a digital signal.

The acoustic signal is continuous data in the depth direction configuring the RF signal after the delay-and-sum process has been performed thereon. The depth direction is a direction in which a transmission ultrasound signal travels from the surface of a body of a subject toward the inside of the body of the subject. The acoustic signal is for example formed from a plurality of frames in the transducer arrangement direction and in an ultrasound transmission direction perpendicular thereto. An acoustic signal acquired through a single ultrasound scan is referred to as a frame acoustic signal. The term “frame” is used to express a unit of a group of signals necessary in order to construct a single ultrasound image.

The transceiver 103 successively repeats the transmission process and the reception process.

3. Image Recording Circuitry 151

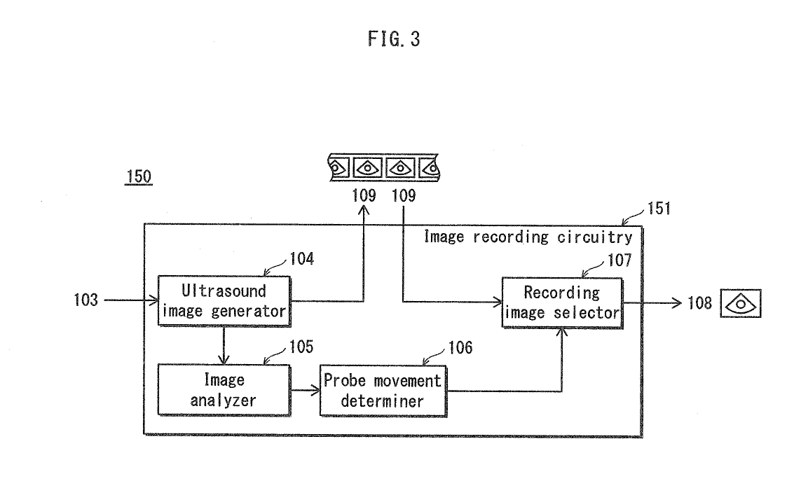

The following explains configuration of the image recording circuitry 151 of the ultrasound diagnostic apparatus 150 pertaining to Embodiment 1. FIG. 3 is a block diagram illustrating configuration of the image recording circuitry 151 of the ultrasound diagnostic apparatus 150. As described above, the image recording circuitry 151 of the ultrasound diagnostic apparatus 150 includes the ultrasound image generator 104, the image analyzer 105, the probe movement determiner 106 and the recording image selector 107.

(1) Ultrasound Image Generator 104

The ultrasound image generator 104 is a circuit that generates ultrasound images based on reflected ultrasound from the subject acquired by the probe 101. The ultrasound images are B-mode ultrasound images in which the strength of an ultrasound reception signal is expressed by luminance. The following explains a case where the examiner acquires reflected ultrasound while moving the probe along the body surface of the subject. Note that the probe 101 is not necessarily moved along a straight line. That is, the following explanation is applicable to the case where the examiner moves the probe 101 along a curved line.

The ultrasound image generator 104 generates an ultrasound image as a single frame by converting each acoustic signal of each frame into a luminance signal corresponding to strength of the acoustic signal, and by subsequently performing coordinate conversion on the luminance signal to an orthogonal coordinate system The ultrasound image generator 104 sequentially performs this processing for each frame, and outputs the generated ultrasound images to the frame memory 109 and the image analyzer 105 via the controller 102. Specifically, the ultrasound image generator 104 performs filter processing on reflected ultrasound, and then performs envelope detection. Furthermore, the ultrasound image generator 104 performs logarithmic conversion and gain control on a signal resulting from the envelope detection, thereby generating ultrasound images. The ultrasound images as frames generated by the ultrasound image generator 104 are sequentially stored in the frame memory 109 each time ultrasound scan is performed.

(2) Image Analyzer 105

The image analyzer 105 receives reflected ultrasound from the ultrasound image generator 104 and ultrasound images from the ultrasound image generator 104 or the frame memory 109, and analyzes the images to calculate motion information of each image area in each ultrasound image. The “motion information” is composed of the amount of movement detected from the same image area of a plurality of ultrasound images of the inside of the subject's body, and the direction of the movement of the image area.

The image analyzer 105 analyzes each ultrasound image, and extracts the motion information of each image area. The motion information is obtained by comparing at least two ultrasound images with each other, and detecting the direction of the movement in each image area of each ultrasound image and the amount of the movement.

The detection of the direction and the amount of the movement can be implemented by using conventional technology. For example, block matching may be used.

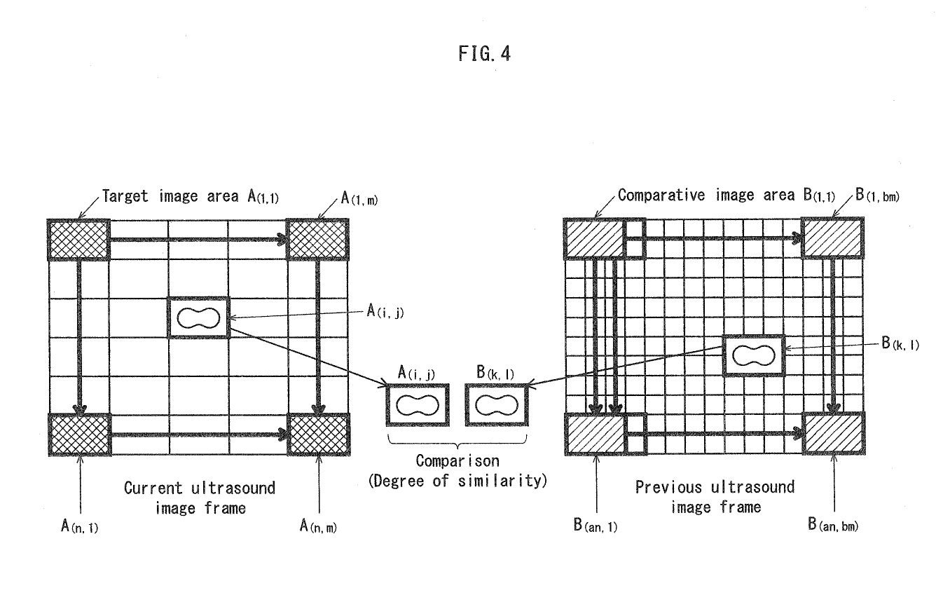

FIG. 4 is a diagram illustrating motion information extraction from each ultrasound image according to block matching, performed by the image analyzer 105 of the ultrasound diagnostic apparatus 150. In block matching, first, the image analyzer 105 divides the current ultrasound image, which is to be analyzed, into small rectangular target image areas A_{(1,1)} through A_{(n,m)} which are called “blocks”, and calculates the degree of similarity between each of the target image areas A_{(i,j)} (1≦i≦n, 1≦j≦m) with the comparative image areas B_{(1,1)} through B_{(an,bm)} (where a and b are coefficients) of an ultrasound image obtained in the past, and then detects, from the two ultrasound images, a pair of image areas having a high degree of similarity. Specifically, the image analyzer 105 calculates the difference in luminance between each of the target image areas A_{(i,j)} (1≦k≦n, 1≦j≦m) and each of the comparative image areas B_{(k,l)} (1≦k≦an, 1≦l≦bm), and calculates the cumulative value of the difference in luminance for each of the comparative image area B_{(k,l)}. The image analyzer 105 determines the comparative image area B_{(k,l)} with the smallest cumulative value as the image area having a high degree of similarity and correlation with the target image area A_{(i,j)}. That is, when an image area has a small cumulative value of the difference in luminance, this image area has a high degree of similarity and correlation. The image analyzer 105 calculates the direction and the amount of the movement in the image area A_{(i,j)} from the positional relationship with the comparative image area B_{(k,l)} that has a high degree of correlation with the image area A_{(i,j)}.

As an alternative to the block matching, optical flow detection may be used for example. An optical flow is a velocity vector between a pixel of a frame at one time point and a pixel of another frame at another time point. In an optical flow detection using a gradient method, the direction of change in luminance gradient is calculated between frames on the assumption that local areas of an image show only a small change in brightness and the luminance value is constant before and after the movement, and the optical flow is detected based on the direction. Changes in position of feature points in the image are calculated based on the optical flow.

In any of the aforementioned methods, the frames from which the amount of movement is detected are not necessarily adjacent frames, and may be frames with a given interval therebetween. Details of the method of detecting the direction and amount of the movement are described below.

The motion information of each image area so obtained is output to the probe movement determiner 106 via the controller 102.

(3) Probe Movement Determiner 106

The probe movement determiner 106 is a circuit that determines the movement of the probe 101 which is either stationary or moving, based on the motion information of each image area of each ultrasound image, calculated by the image analyzer 105. The following explains the case where the probe 101 moves along a direction that is substantially parallel with the surface of the subject's body. Note that the subject of the probe movement determination is not limited to the movement of the probe 101.

The probe movement determiner 106 performs motion information analysis and probe movement determination.

In the motion information analysis, the probe movement determiner 106 merges the motion information of each image area of an ultrasound image, and thereby determines the movement in the entire ultrasound image. Here, a motion direction histogram is created from the motion information so acquired, and the movement of the entire ultrasound image is determined from the histogram, based on the occurrence frequency of each of the ranges (i.e. bins) indicating a motion direction of the image areas (i.e., the number of image areas belonging to each motion direction).

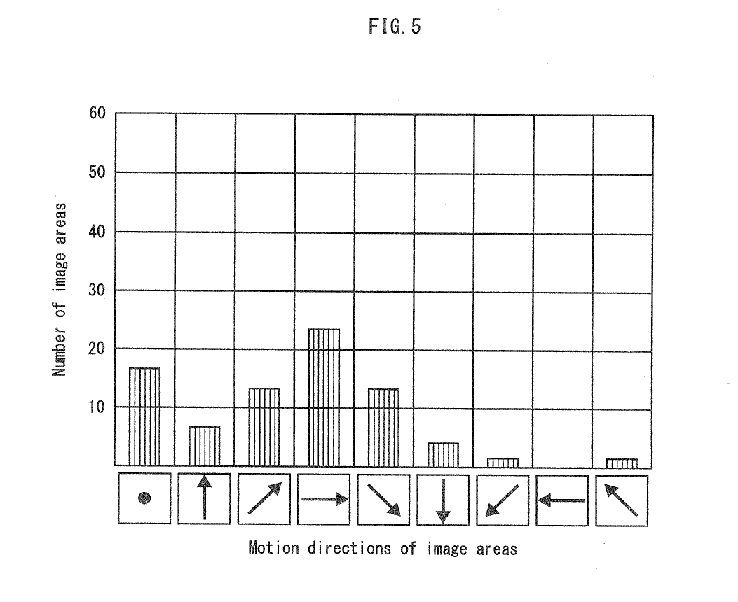

FIG. 5 is an example of a motion direction histogram used by the probe movement determiner 106 for determining the movement of the probe 101, which is either stationary or moving. This histogram can be created by, for example, classifying the image areas into nine ranges (i.e., bins), each indicating movement along one of eight radial directions from the center point or the stationary state, and counting the number of the image areas belonging to each range (i.e., bin). Here, the stationary state includes both the case where the amount of movement is zero and the case where the amount of movement is no greater than the upper limit (hereinafter, “reference value”) below which the movement is regarded as “stationary”. Furthermore, the angle formed by adjacent two of the directions is not necessarily 45°, and may form a different angle within a predetermined angle range such as ±45°. In the example shown in FIG. 5, the number of image areas belonging to the range (i.e., bin) “•”, which indicates the stationary state, is approximately 16, and the number of image areas belonging to the range (i.e., bin) “→”, which indicates the angle corresponding to the direction toward the right, is approximately 24.

In the probe movement determination, the probe movement determiner 106 determines movement of the probe 101, which is either stationary or moving, occurring at the acquisition of the reflected ultrasound from which the ultrasound images are generated. More specifically, each ultrasound image is generated from the corresponding portion of the reflected ultrasound. Therefore, each ultrasound image shows movement of the probe occurring at the acquisition of the corresponding portion of the reflected ultrasound. In the following description, however, for the sake of simplification, the movement of the probe shown in an ultrasound image is referred to as “the movement of the probe ‘at the generation of the ultrasound image’”, for example.

The movement of the probe 101 can be determined from, for example, the occurrence frequency of the range (i.e., bin) corresponding to “stationary”, from among the ranges (i.e., bins) of the histogram. When the number of the image areas belonging to the range (i.e., bin) corresponding to “stationary” is the largest and this number is no less than the threshold value A of the occurrence frequency (below which the probe 101 is considered as moving), such a frame is considered to indicate that the probe 101 was not moving along the surface of the subject's body. Otherwise, the frame is considered to indicate that the probe 101 was moving along the surface of the subject's body.

The result of the determination by the probe movement determiner 106 is output to the recording image selector 107 via the controller 102.

(4) Recording Image Selector 107

The recording image selector 107 is a circuit that determines whether an ultrasound image is to be recorded or not, based on the result of the determination by the probe movement determiner 106, i.e., the movement of the probe 101, which is either stationary or moving, determined by the probe movement determiner 106. According to the result of the determination by the probe movement determiner 106, the recording image selector 107 specifies the ultrasound image determined as showing that the probe 101 was stationary at the generation of the ultrasound image, and determines the ultrasound image as an ultrasound image to be recorded. This is because an ultrasound image generated while the probe 101 is moving is likely to include motion blurs and is accordingly not likely to be an ultrasound image to be recorded. As shown in FIG. 3, the recording image selector 107 acquires the ultrasound images as frames from the frame memory 109, and performs the same processing as to each of the frames. When the ultrasound image to be recorded as a recording image is selected, the recording image selector 107 outputs the selected image to be recorded, to the ultrasound image recorder 108 via the controller 102.

Note that the following explains an example case where only one ultrasound image is to be recorded. However, the number of ultrasound images to be recorded is not limited to one, and may be plural. In other words, the present disclosure is applicable to the case where a plurality of still images are to be recorded, or the case where a video composed of a plurality of frames is to be recorded.

4. Ultrasound Image Recorder 108

The ultrasound image recorder 108 is a circuit that acquires via the controller 102 and records the ultrasound images generated by the ultrasound image generator 104, the analysis information generated by the image analyzer 105, the determination information generated by the probe movement determiner 106, and the recording image determined by the recording image selector 107. The ultrasound image recorder 108 may be implemented as, for example, a flexible disk, a hard disk, a CD-ROM, an MO, a DVD, a DVD-ROM, a DVD-RAM, or a semiconductor memory. Whether or not the ultrasound image recorder 108 is provided inside the ultrasound diagnostic apparatus 150 is optional.

5. Frame Memory 109

The frame memory 109 is a buffer for receiving, via the controller 102, and sequentially storing ultrasound images generated by the ultrasound image generator 104 each time ultrasound scan is performed. The frame memory 109 is a computer readable recording medium, and may be implemented as a semiconductor memory or a hard disk. Note that the frame memory 109 may be configured as a buffer for storing some of the ultrasound images received from the ultrasound image recorder 108.

<Operations>

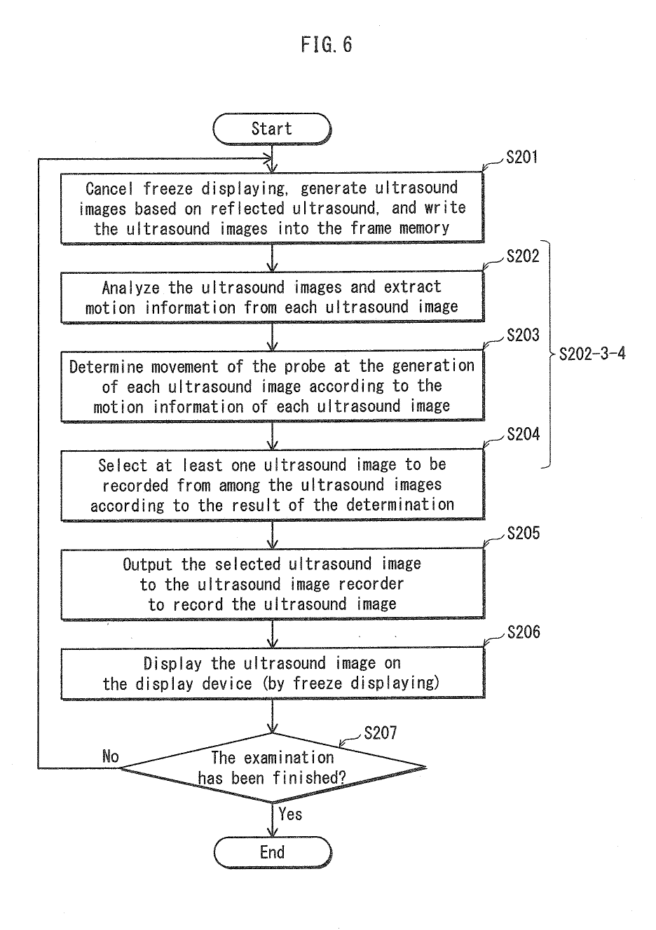

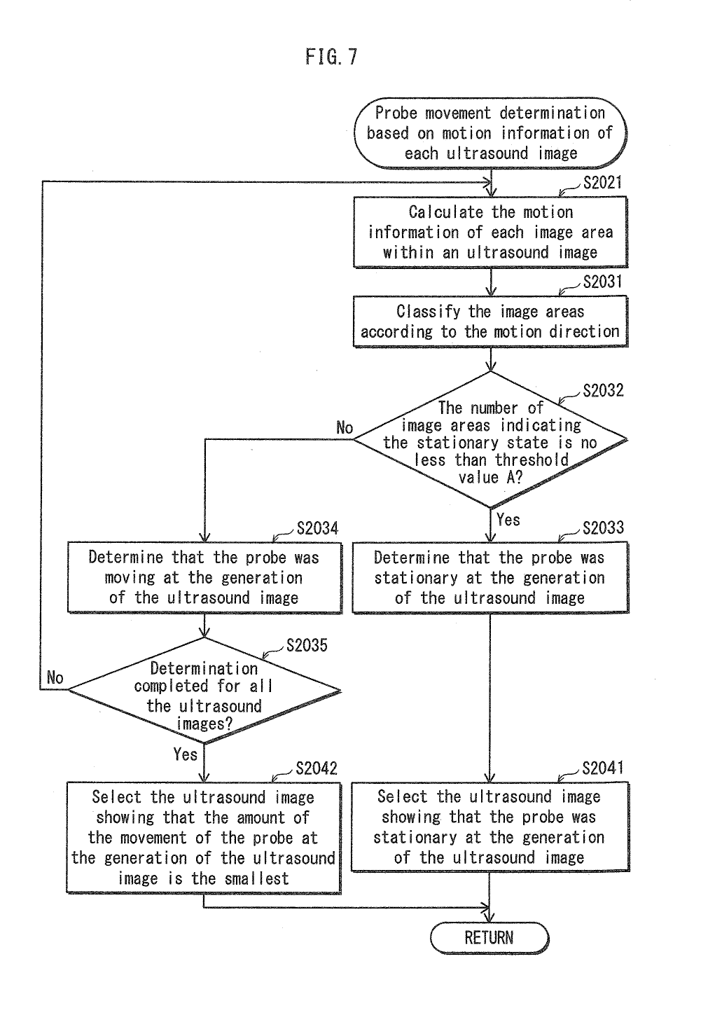

Operations of the ultrasound diagnostic apparatus 150 pertaining to Embodiment 1 are described below. FIG. 6 is a flowchart illustrating operations of the ultrasound diagnostic apparatus 150 when outputting an image to be recorded. FIG. 7 is a flowchart showing details of Steps S202, S203 and S204 of the flowchart shown in FIG. 6.

1. Step S201

At Step S201, the ultrasound image generator 104 generates ultrasound images. Specifically, the transceiver 103 transmits ultrasound to the subject via the probe 101, receives reflected ultrasound from the subject via the probe 101, and outputs reception signals. The ultrasound image generator 104 generates ultrasound images by processing the reception signals based on the reflected ultrasound received by the transceiver 103, and writes the ultrasound images so generated into the ultrasound image recorder 108. Also, the controller 102 displays the ultrasound images so generated on the display device 110 according to a freeze cancellation instruction input by the examiner from the operation console 111. Note that the generation of the ultrasound images may be performed sequentially in a time series (hereinafter, a unit of ultrasound images generated at a time is referred to as a frame).

In the following explanation of the ultrasound diagnostic apparatus 150, it is assumed that the examiner acquires the reflected ultrasound while moving the probe 101 on the subject, and thereby acquires a plurality of ultrasound images as frames. Note that the orientation and the position of the probe 101 is determined by the examiner in order to obtain appropriate ultrasound images of the target part. For this purpose, the examiner moves the probe 101 to acquire a desirable ultrasound image of the target part while referencing the ultrasound images that are provided sequentially. In the following explanation of the ultrasound diagnostic apparatus 150, it is assumed that the examiner stops moving the probe 101 when successfully acquiring a desirable ultrasound image of the target part. Note that the ultrasound image generator 104 generates ultrasound images regardless of whether the probe 101 is being moved or not.

2. Step S202

At step S202, the image analyzer 105 analyzes the ultrasound images, and detects motion information of each image area of each image. For example, the image analyzer 105 analyzes a plurality of consecutive frames such as frames at different time points to detect image areas indicating a movement, and calculates the direction and amount of the movement. According to the ultrasound diagnostic apparatus 150, the motion information indicating the direction and the amount of movement in each image area of each ultrasound image is acquired by the block matching illustrated in FIG. 4. When the motion information of each image area within a single ultrasound image as a frame is acquired (Step S2021), the process proceeds to Step S2031. The acquisition of the motion information is performed until the determination completes as to every ultrasound image generated by the ultrasound image generator 104.

3. Step S203

At Step S203, the probe movement determiner 106 analyzes the motion information acquired at Step S202, and determines the movement of the probe 101, which is either stationary or moving, occurring at the generation of the ultrasound image. The movement of the probe 101 is determined as to each frame. In particular, a frame showing that the probe 101 was stationary is specified.

At Step S203, first, the probe movement determiner 106 classifies the image areas of a single frame into any one of the motion directions (Step S2031). The classification of the image areas according to the motion direction is performed by using the histogram as shown in FIG. 5. In other words, the probe movement determiner 106 classifies each of the image areas of the ultrasound image into one of nine ranges (i.e. bins), each indicating movement along one of eight radial directions from the center point, or the stationary state. At this stage, the probe movement determiner 106 determines that an image area indicates the stationary state when the amount of movement of the image area is no greater than the reference value.

Next, the probe movement determiner 106 determines, with respect to the single ultrasound image, whether the number of image areas that indicate the stationary state is no less than the threshold value A (Step S2032). When the number of image areas with an amount of movement no greater than the reference value is no less than the threshold value A, the probe movement determiner 106 determines that the probe 101 was stationary at the generation of the ultrasound image (Step S2033), and otherwise the probe movement determiner 106 determines that the probe 101 was moving at the generation of the ultrasound image (Step S2034).

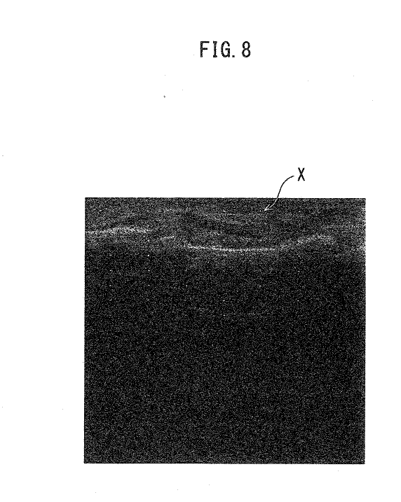

The following explains the probe movement determination performed at Step S2032, with reference to FIGS. 8, 9, 10 and 11.

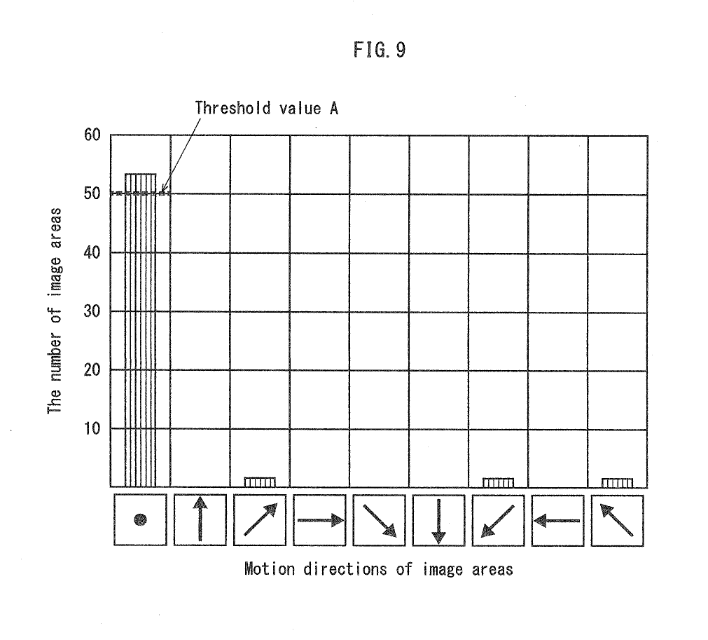

FIG. 8 is a diagram illustrating motion information of each image area when the image analyzer 105 determines that the probe 101 was stationary, overlaying arrow images X on an ultrasound image. The directions of the arrow images X indicate the motion directions of the image areas, and the lengths of the arrow images X indicate the amounts of movement of the image areas. In FIG. 8, the arrow images X are short, and for almost all the image areas, the amount of movement is no greater than the reference value, which indicates the stationary state. The movement of the probe 101 is determined from a motion direction histogram using information corresponding to the arrow images X shown in FIG. 8. FIG. 9 is an example of a motion direction histogram showing motion directions detected from the ultrasound image shown in FIG. 8. In the example shown in FIG. 9, the number of image areas belonging to the range (i.e., bin) corresponding to the stationary state is no less than the threshold value A. In this case, it is to be determined at the probe movement determination (Step S203) that the probe 101 was stationary.

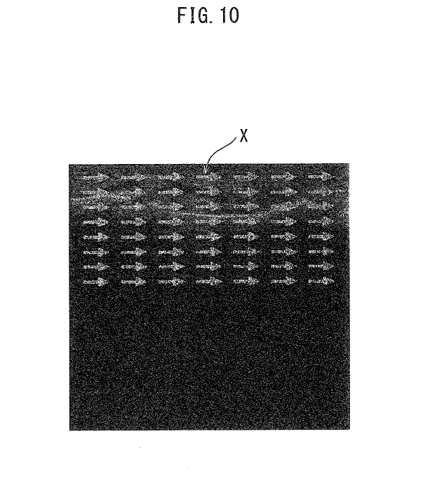

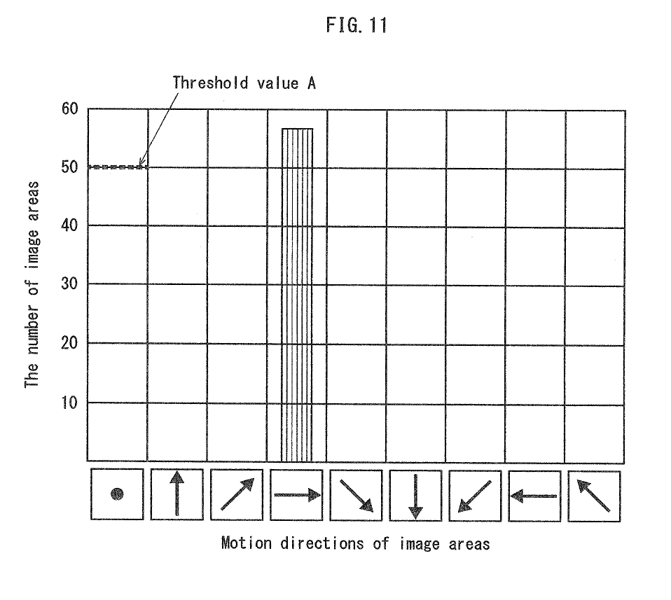

FIG. 10 is a diagram illustrating motion information of each image area when the image analyzer 105 determines that the probe 101 was moving, overlaying the arrow images X on an ultrasound image. In FIG. 10, the lengths of the arrow images X with respect to all the image areas are greater than the reference value, and the movement of all the image areas is directed towards the right of the screen. In the probe movement determination, it is determined that the probe 101 moves along the surface of the subject's body. FIG. 11 is an example of a motion direction histogram showing motion directions detected from the ultrasound image shown in FIG. 10; In the example shown in FIG. 11, the number of image areas belonging to the range (i.e., bin) corresponding to the stationary state is less than the threshold value A, and accordingly it is determined that the probe 101 was moving.

As explained above, in the case of the example shown in FIG. 10, in which it is determined that the probe 101 was moving, all the image areas show a movement towards the same direction. In contrast, in the case of the example shown in FIG. 8, the amount of movement is no greater than the reference value with respect to almost all of the image areas, and the number of image areas belonging to the range (i.e., bin) corresponding to the stationary state is no less than the threshold value A. Whether the probe 101 was stationary or moving can be determined from the ultrasound image by dividing the ultrasound image into image areas and counting the number of image areas belonging to each of the ranges (i.e., bins) indicating the motion directions.

4. Step S204

At Step S204, the recording image selector 107, at least one of the ultrasound images is selected as an image to be recorded, based on the result of the determination at Step S203. When it is determined that an ultrasound image shows that the probe 101 was stationary at the generation of the ultrasound image (Step S2033), the ultrasound image is selected as the image to be recorded (Step S2041). This operation allows for, based on the probe movement determination, selection of an ultrasound image showing that the probe 101 was stationary at the generation of the ultrasound image, and accordingly the examiner can easily perform the freeze operation for selecting an d recording the ultrasound image to be recorded.

On the other hand, when it is determined that the probe 101 was moving at the generation of the ultrasound image (Step S2034), a determination is made as to whether the probe movement determination has been completed for all the ultrasound images generated by the ultrasound image generator 104 (Step S2035). When the probe movement determination has not been completed for all the ultrasound images, the process returns to Step S2021, and the motion information is acquired from the next ultrasound image. When the probe movement determination has been completed for all the ultrasound images, the ultrasound image showing that the amount of the movement of the probe 101 occurring at the generation thereof is the smallest is selected as the image to be recorded (Step S2042). Through this operation, even when no ultrasound image is determined as showing that the probe 101 was stationary at the generation of the ultrasound image, an ultrasound image generated when the probe 101 was in the state that is closest to the stationary state can be selected as the image to be recorded.

5. Step S205

At Step S205, the ultrasound image selected at Step S204 is output to the ultrasound image recorder 108 via the controller 102 and then stored in the ultrasound image recorder 108.

6. Step S206

At Step S206, freeze displaying is performed, by which the ultrasound image selected at Step S204 is continuously displayed on the display device 110 by the controller 102. This operation allows for an easier freeze operation by which the image to be recorded, selected according to the probe movement determination, is continuously displayed on the display device 110.

After Step S206, the process may be returned to Step S201 to repeat the generation of ultrasound images according to an instruction from the examiner input at Step S207. For example, when it is determined at Step S2035 that the determination has been completed as to all the ultrasound images, and the ultrasound image showing that the amount of the movement of the probe 101 occurring at the generation thereof is the smallest is selected at Step S2042 as the image to be recorded, the examiner may be allowed to determine at Step S207 to continue the examination so that the process returns to Step S201 to perform the generation of the ultrasound images again.

If this is the case, the ultrasound image recorded at Step S206 may be displayed in a different area than the area of the screen of the display device 110 in which the ultrasound image generated at Step S201 is displayed. This operation allows the examiner to check the ultrasound image that has been actually recorded.

<Effects>

As described above, the ultrasound diagnostic apparatus 150 pertaining to Embodiment 1 includes the image recording circuitry 151 that includes: the ultrasound image generator 104 that generates a plurality of ultrasound images of the inside of the subject's body based on the reflected ultrasound; the probe movement determiner 106 that determines, based on motion information obtained by analyzing the plurality of ultrasound images, movement of the probe occurring at generation of each of the plurality of ultrasound images; the recording image selector 107 that selects at least one ultrasound image from among the plurality of ultrasound images according to the determination performed by the probe movement determiner; and the ultrasound image recorder 108 that records therein said at least one ultrasound image selected by the recording image selector 107.

With this configuration, at least one ultrasound image is selected and recorded based on the result of the probe movement determination performed by the probe movement determiner 106. Therefore, the freeze operation, by which the ultrasound image to be recorded is selected and recorded, can be easily performed. As a result, the examiner can record the image with both hands full, for example. Also, the ultrasound image thus selected and recorded is an image generated when the probe 101 was stationary, and is not likely to include motion blurs.

<Modification 1>

Embodiment 1 above is an example case where, when the probe movement determiner 106 determines that the amount of movement in each image area of an ultrasound image is no greater than the reference value and the number of image areas belonging to the range (i.e., bin) corresponding to the stationary state is no less than the threshold value A, the ultrasound image is recorded as an image showing that the probe 101 was stationary at the generation of the ultrasound image. However, the probe movement determiner 106 may determine the movement of the probe 101 by additionally taking into consideration the motion information of each image area. For example, the probe movement determiner 106 determine the movement of the probe 101 by distinguishing the movement of the entire ultrasound image and the movement of each image area from each other.

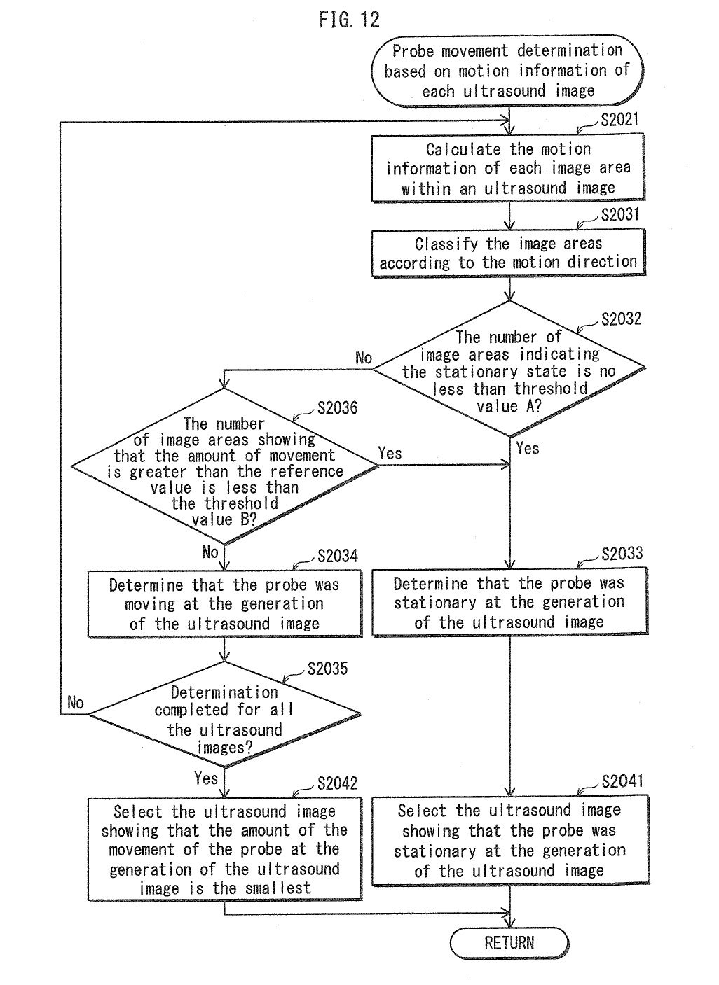

FIG. 12 is a flowchart showing details of operation pertaining to Modifications 1, in which Steps S202, S203 and S204 of the flowchart shown in FIG. 6 are modified. In FIG. 12, operations performed at Steps S2021, S2031 and S2032, and operations performed after it is determined at Step S2032 that the number of image areas showing the stationary state is no less than the threshold value A, are the same as Embodiment 1. Modification 1 is different from Embodiment 1 in that when it is determined that the number of image areas showing the stationary state is less than the threshold value A, another determination is performed as to whether the number of image areas showing a movement in each direction is less than a threshold value B, which is smaller than the threshold value A.

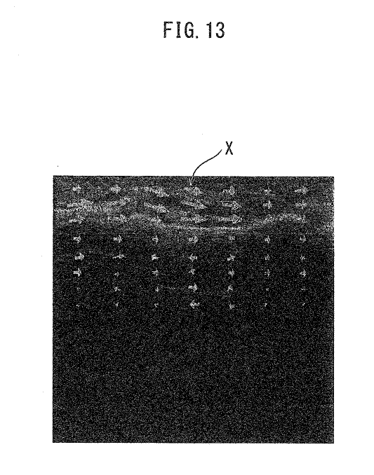

FIG. 13 is a diagram illustrating motion information of each image area when the image analyzer 105 determines that inner tissues of the subject's body are moving, overlaying arrow images X on an ultrasound image. FIG. 13 is an ultrasound image showing an example case where inner tissues of the subject's body are moving, and specifically is an ultrasound image of a joint of a finger being bent and stretched. In this example, mainly the upper areas that corresponds to the flexor tendon is moving, and the arrows on other areas show no constant movement. As shown in FIG. 13, each arrow has a different length depending on the image area, and the arrows in the upper image areas are relatively long. Regarding the motion direction, the arrows in the upper image areas are directed towards the right, whereas the arrows in lower image areas are not directed toward the same direction. In this case, the probe movement determiner 106 preferably does not determine that the probe 101 was moving and, instead determines that the probe 101 was stationary and the inner tissues of the subject's body were moving.

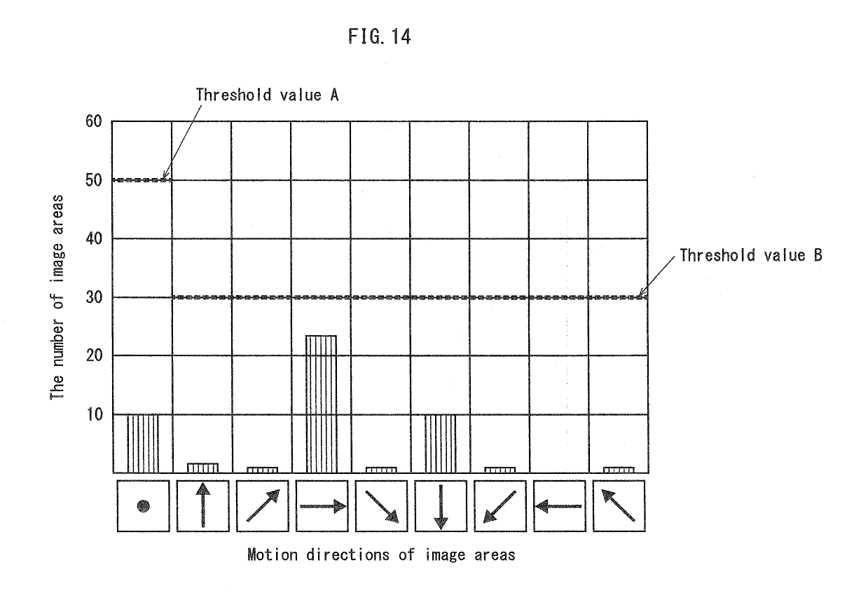

FIG. 14 is an example of a motion direction histogram showing motion directions detected from the ultrasound image shown in FIG. 13; As explained above, in the case of the example shown in FIG. 10 in which it is determined that the probe 101 was moving, all the image areas show movement towards the same direction. In contrast, in FIG. 14, the image areas belong to several ranges (i.e. bins) corresponding to different directions. Such a result is determined as showing that the inner tissues of the subject's body captured in the image were moving. In such a case, it is impossible to determine that the probe 101 was stationary, because the number of image areas belonging to the range (i.e. bin) corresponding to the stationary state is less than the threshold value A. In response to this problem, Modification 1 additionally has Step S2036, at which it is determined that an ultrasound image shows that the probe 101 was stationary at the acquisition of the reflected ultrasound when the number of its image areas showing that the amount of movement is greater than the reference value is less than the threshold value B (smaller than the threshold value A) with respect to every one of the plurality of motion angle ranges. Through this operation, the movement of the entire ultrasound image and the movements of the image areas of the ultrasound image can be distinguished from each other. In other words, the movement of the probe 101 and the movements of the inner tissues of the subject's body can be distinguished from each other. As a result, the movement of the probe 101 can be determined independently from the movements of the inner tissues of the subject's body.

<Modification 2>

In the embodiment above, the probe movement determination is a determination as to whether the probe 101 was moving along the surface of the subject's body. However, another probe movement determination may be performed alone or together with the aforementioned probe movement determination. For example, a vertical movement determination may be performed to determine whether the probe 101 was moving away from the body surface. This determination can be performed based on the number of image areas belonging to the range (i.e. bin) corresponding to the downward movement and on the amount of the movement. Generally, an ultrasound image is displayed such that the upper side corresponds the body surface (closer to the probe 101) and the lower side corresponds to the inside of the body. The downward movement of each image area of the ultrasound image shows that the inner tissues of the subject's body are moving away from the probe 101. Thus, whether the probe 101 was moving away from the body surface can be determined by the vertical movement determination.

Modification 2 shows an example case where the probe movement determiner 106 determines whether the probe 101 was moving in a direction that is perpendicular to the body surface, and the result is used in the recording image selection.

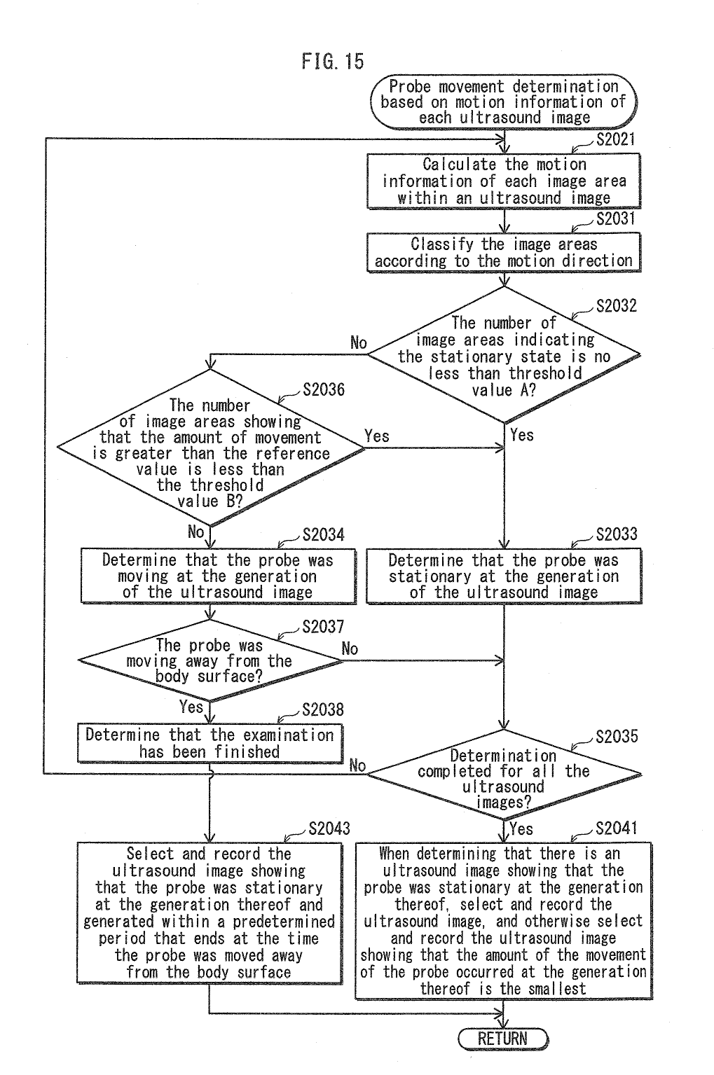

FIG. 15 is a flowchart showing details of the operation pertaining to Modification 2, in which Steps S202, S203 and S204 of the flowchart shown in FIG. 6 are modified. In FIG. 15, operations performed at Steps S2021, S2031, S2032, S2033, S2034 and S2036 are the same as Modification 1. Modification 2 is different from Modification 1 in that when it is determined at Step S2034 that the probe 101 was moving, a determination is performed as to whether the probe 101 was moving away from the body surface (S2037), and when it is determined that the probe 101 was moving away from the body surface, it is then determined that the examination has been finished (S2038). If this is the case, the ultrasound image to be recorded is an image showing that the probe 101 was stationary and generated within a predetermined period that ends at the time the probe 101 was moved away from the body surface (S2043). In other words, the operation of moving the probe 101 away from the body surface is considered as a trigger of finishing the examination, and a frame that was generated earlier than the start of the vertical movement and that is determined to be an image showing that the probe 101 was stationary is selected as an ultrasound image to be recorded. This method is based on results of inventor's observation indicating that examiners often move the probe away from the body surface immediately after acquiring a desired ultrasound image to be recorded. With this method, the operation of moving the probe 101 away from the body surface is regarded as a trigger of finishing the examination, and accordingly the image that the examiner possibly wish to record can be selected in response to the operation.

When it is determined at Step S2033 that the probe 101 was stationary, a determination is made as to whether the probe movement determination has been performed for every one of the ultrasound images generated by the ultrasound image generator 104 (Step S2035). When the probe movement determination has not been completed for every one of the ultrasound images, the process returns to Step S2021, and the motion information is acquired from the next ultrasound image. When the probe movement determination has been completed for every one of the ultrasound images, the ultrasound image showing that the probe 101 was stationary at the acquisition of the reflected ultrasound is selected as the image to be recorded (Step S2041).

When it is determined that the probe 101 was moving (S2034) but was not moving away from the body surface (No at Step S2037), and when the probe movement determination has been completed for every one of the ultrasound images, an ultrasound image showing that the probe 101 was stationary, if any, is selected from among the ultrasound images, and if there is no such an ultrasound image, the ultrasound image showing that the amount of the movement of the probe 101 occurring at the acquisition of the reflected ultrasound is the smallest is selected as the image to be recorded (S2041).

<Other Modifications>

(1) The embodiment above shows an example case where the probe movement determiner 106 determines, as to each of the ultrasound images, whether the ultrasound image shows the probe 101 was stationary at the acquisition of the reflected ultrasound, based on the amount of movement in each of the image areas of the ultrasound image, thereby selecting an ultrasound image to be recorded. In the probe movement determination, however, the length of the period for which the probe 101 was stationary may be measured in order to determine whether the length of the period exceeds a predetermined length. This method is based on results of inventor's observation indicating that examiners spend a relatively long time for viewing an ultrasound image to be recorded, and accordingly the probe 101 is maintained stationary for a relatively long period. It is preferable that the length of the period is five seconds to ten seconds, for example. In addition, the frequency of selecting an image to be recorded may be changeable by the examiner through operation of the probe 101. In other words, when the examiner wishes to select an ultrasound image as an image to be recorded, the examiner can select the image by maintaining the probe 101 stationary for the predetermined period. In this case, the length of the period may be adjustable by the examiner.

(2) In the explanation above, the probe movement determination is performed based on the occurrence frequencies obtained from the motion direction histogram. However, other information may be used instead. For example, the amounts of movement may be averaged for each of the ranges (i.e. bins) corresponding to the motion directions in the motion direction histogram, and may be used for the probe movement determination together with the above-described occurrence frequency of each range. This method allows for probe movement determination without an influence of minor movements.

Also, it is not always necessary to generate a motion direction histogram. For example, an average motion vector for the entire ultrasound image may be calculated, and the movement of the probe 101 may be determined based on the direction and magnitude of the vector.

### Embodiment 2

The following explains an ultrasound diagnostic apparatus 152 pertaining to Embodiment 2 with reference to the drawings.

<Overall Configuration>

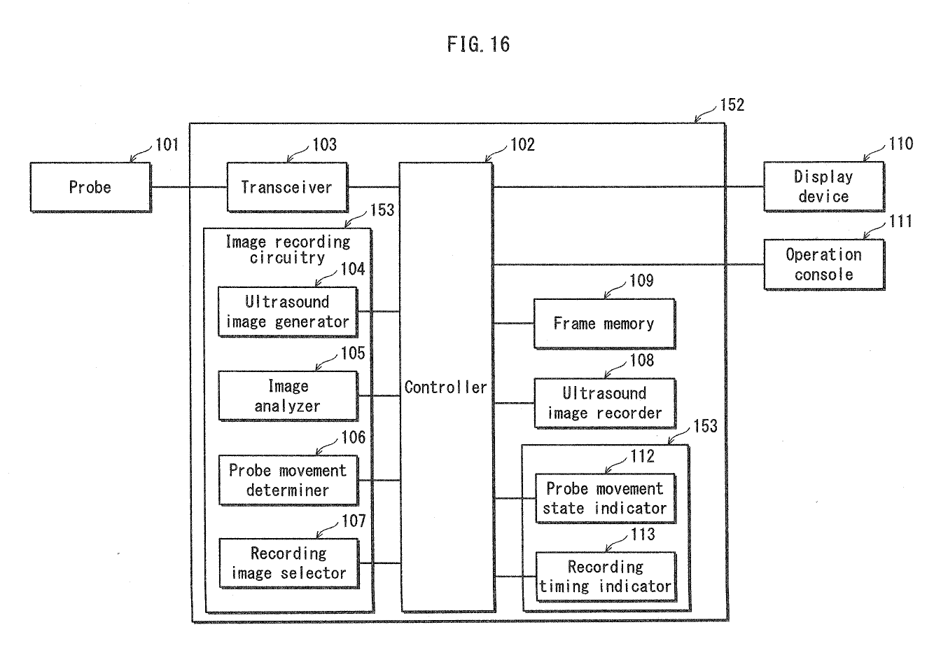

Operations of an ultrasound diagnostic system including the ultrasound diagnostic apparatus 152 pertaining to Embodiment 2 are described below. FIG. 16 is a block diagram showing configuration of an ultrasound diagnostic system in which the ultrasound diagnostic apparatus 152 pertaining to Embodiment 2 is included.

The ultrasound diagnostic apparatus 152 shown in FIG. 16 is different from the ultrasound diagnostic apparatus 150 shown in FIG. 2 in that the image recording circuitry 153 additionally includes a probe movement state indicator 112 and a recording timing indicator 113. In FIG. 16, elements of configuration that are the same as in FIG. 2 are labeled using the same reference signs and explanation thereof is omitted.

In the ultrasound diagnostic apparatus 150 pertaining to Embodiment 1, an ultrasound image is recoded according to the result of the probe movement determination. In some cases, however, an image is recorded with timing that is not intended by the examiner due to such automatic recording. For example, when the examiner is performing an examination while moving the probe 101 slowly, if the probe 101 is mistakenly determined to be stationary, an image is automatically selected and recorded (hereinafter, “automatic recording”). In such cases, it is unknown to the examiner why the image was automatically recorded, and accordingly a problem arises in an ultrasound examination or recording of an ultrasound image. In response to this problem, the ultrasound diagnostic apparatus 152 visualizes the result of the probe movement determination, and notifies the examiner of the recording timing. Specifically, the probe movement indicator 112 is a circuit that notifies the examiner of the result of the probe movement determination. The recording timing indicator 113 is a circuit that notifies the examiner of the recording timing.

The display device 110 displays images generated by the probe movement indicator 112 and the recording timing indicator 113 in addition to the ultrasound images, information of the probe movement determination, and an image to be recorded.

<Images Presented by Probe Movement Indicator 112 and Recording Timing Indicator 113>

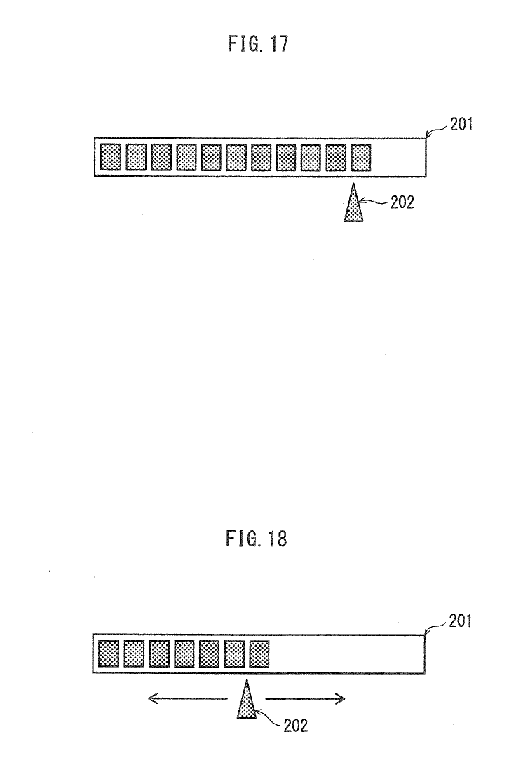

FIG. 17 is a schematic diagram illustrating images provided by the probe movement state indicator 112 and the recording timing indicator 113 of the ultrasound diagnostic apparatus 152 pertaining to Embodiment 2. As shown in FIG. 17, the image 201 provided by the probe movement indicator 112 is, for example, an indicator bar. The probe movement indicator 112 shows that the probe 101 is determined to be in the stationary state by elongating the bar according to the duration of the stationary state. The image 202 provided by the recording timing indicator 113 is, for example, an indicator arrow, and is displayed together with the image 201 provided by the probe movement indicator 112. When an image is to be recorded at the point the duration of the stationary state of the probe 101 reaches a fixed time length, the image 202 is displayed to indicate the fixed time length of the stationary state. Accordingly, an image is recorded when the length of the indicator bar provided by the probe movement indicator 112 exceeds the length indicated by the recording timing indicator 113. This configuration allows the examiner to know the timing of the automatic recoding by checking whether the length of the indicator bar has exceeded the length indicated by the indicator arrow.

Note that the length indicated by the indicator arrow as the image 202 provided by the recording timing indicator 113 may be determined by the examiner. FIG. 18 is a schematic diagram illustrating the image 202 provided by the recording timing indicator 113 of the ultrasound diagnostic apparatus 152 pertaining to Embodiment 2. For example, as shown in FIG. 18, the image 202 may be movable to the left or the right according to an instruction input from the operation console 111. If this is the case, the recording image selector 107 is to be configured to change the condition for the determination according to the instruction. In the example shown in FIG. 18, the threshold of the duration of the stationary state has been reduced by an instruction from the operation console 111. As a result, the recording image selector 107 performs the determination according to the timing newly indicated by the recording timing indicator 113. In other words, the examiner can adjust the recording timing.

With this configuration, the examiner is notified of the result of the probe movement determination, and the examiner can change the timing of the automatic recording.

<Other Modifications>

According to the ultrasound diagnostic apparatus 150 pertaining to the above embodiments, the ultrasound image recorder 108 and the frame memory 109, which are examples of a storage device, are included in the ultrasound diagnostic apparatus 150. However, the storage devices are not limited to such a configuration and may alternatively be a semiconductor memory, a hard disk drive, an optical disk drive, a magnetic storage device, or the like that is connected to the ultrasound diagnostic apparatus 150 from externally thereto.

Furthermore, although an example of configuration is explained in which the ultrasound probe 101 and the display device 110 are connected to the ultrasound diagnostic apparatus 150 from externally thereto, alternatively the aforementioned elements may all be integrated into the ultrasound diagnostic apparatus 150.

Furthermore, the ultrasound probe 101 may also include an inclination angle measurer such as an angle sensor and an inclination angle of the ultrasound probe 101 which is measured may be recorded in examination results in combination with corresponding disease progression scores.

In the embodiment described above, the probe 101 includes a plurality of piezoelectric elements that are arranged in a one dimensional array. However, the probe 101 is not limited to such a configuration. For example, the probe 101 may include a two-dimensional transducer in which a plurality of piezoelectric elements are arranged in a two-dimensional array, or an oscillating ultrasound probe that has a plurality of transducers arranged in a one-dimensional array and acquires three-dimensional ultrasound images while mechanically oscillating the transducers. The configuration of the probe 101 may be selected according to the measurement to be performed. For example, in a configuration in which the probe 101 includes piezoelectric elements arranged in a two-dimensional array, irradiation position and direction of a transmitted ultrasound beam can be controlled by adjusting magnitude and timing of voltage application to each of the piezoelectric elements.

Also, the probe 101 may have some of the functions of the transceiver. For example, the probe 101 may generate a transmission electrical signal based on a control signal output from the transceiver for generation of the transmission electrical signal, and may convert the transmission electrical signal to ultrasound. The probe 101 may also convert reflected ultrasound received thereby to a reception electrical signal, and may generate a reception signal based on the reception electrical signal.

Typically process components included in the ultrasound diagnostic apparatus relating to the embodiment are implemented through a large scale integration (LSI) which is a type of integrated circuit (IC). Each of the components may be integrated individually into a single chip. Alternatively, some or all of the components may be collectively integrated into a single chip.

The embodiment was explained for an example in which each block is an independent piece of hardware. However, the blocks included in the ultrasound diagnostic apparatus are not limited to being independent pieces of hardware. For example, functions of each of the blocks may be implemented as necessary through a combination of a CPU and software.

With regards to functional blocks included in the ultrasound diagnostic apparatus, typically a portion or all of the functions of the functional blocks can be implemented through an LSI. Each of the functional blocks may be integrated individually into a single chip. Alternatively, some or all of the functional blocks may be collectively integrated into a single chip. Note that depending on the degree of integration, an LSI may be referred to as an IC, a system LSI, a super LSI, or an ultra LSI.

Furthermore, the method of circuit integration is not limited to an LSI and may alternatively be implemented through a dedicated circuit or a general processor. An FPGA which is programmable after the LSI is manufactured or a reconfigurable processor which allows for reconfiguration of the connection and setting of circuit cells inside the LSI may alternatively be used.

Furthermore, if technology for forming integrated circuits that replaces LSI were to emerge, owing to advances in semiconductor technology or to another derivative technology, the integration of functional blocks may naturally be accomplished using such technology.

Also, a portion or all of the functions of the ultrasound diagnostic apparatus relating to the embodiment may be implemented through execution of a program by a processor such as a CPU.

Furthermore, the present invention may alternatively be implemented as the aforementioned program or as a non-transitory computer recordable recording medium on which the program is recorded. Of course, the aforementioned program can also be distributed through a transfer medium such as the Internet.

Note that block diagrams referred to herein only illustrate one example of division of functional blocks. A plurality of the functional blocks may alternatively be implemented in combination as a single functional block, and likewise each one of the functional blocks may alternatively be divided and implemented as a plurality of separate functional blocks. Also, a portion of the functions of one of the functional blocks may be transferred to any other of the functional blocks. A single piece of hardware or software may be used to process functions of a plurality of functional blocks that have similar functions either in parallel or through a time division method.

Note that the order of steps described above is provided merely for explanation of one specific example of the present invention and such steps may alternatively be performed in a different order. Also, part of one of the aforementioned steps may be performed at the same time as (in parallel to) a different one of the aforementioned steps.

Functions of the ultrasound diagnostic apparatus relating to the embodiment and the modified examples thereof may be at least partially combined. Furthermore, the numerical values given above are intended only for describing a specific example of the disclosure, and do not represent any limitation to the given values.

Of course the present invention also includes various modified examples of the embodiment which are within the scope of modifications that a person having ordinary skill in the art might consider.

### CONCLUSION

As described above, one aspect of the present disclosure is an ultrasound diagnostic apparatus to which a probe 101 is connectable and that records an ultrasound image of the inside of a subject's body based on reflected ultrasound from inside the subject's body acquired by the probe 101 positioned to face a surface of the subject's body, the ultrasound diagnostic apparatus including image recording circuitry 151 that includes: an ultrasound image generator 104 that generates a plurality of ultrasound images of the inside of the subject's body based on the reflected ultrasound; a probe movement determiner 106 that determines movement of the probe 101 occurring at generation of each of the plurality of ultrasound images, based on motion information obtained by analyzing the plurality of ultrasound images; a recording image selector 107 that selects at least one ultrasound image from among the plurality of ultrasound images according to the determination performed by the probe movement determiner 106; and an ultrasound image recorder 108 that records therein said at least one ultrasound image selected by the recording image selector 107. This configuration allows for selection of an ultrasound image based on the probe movement determination, and accordingly the freeze operation for selecting the ultrasound image to be recorded can be easily performed.

When the probe movement determiner 106 determines that the probe 101 was stationary at generation of a given ultrasound image from among the plurality of ultrasound images, the recording image selector 107 may select the given ultrasound image. With this configuration, an ultrasound image that is determined to be an image generated when the probe 101 was stationary is selected as the image to be recorded.

When the probe movement determiner 106 determines that the probe 101 was moving along the surface of the subject's body at generation of every one of the plurality of ultrasound images, the recording image selector 107 may select, from among the plurality of ultrasound images, an ultrasound image showing that the amount of movement of the probe 101 was the smallest. With this configuration, even when no ultrasound image is determined as showing that the probe 101 was stationary at the acquisition of the reflected ultrasound, the ultrasound image showing that the probe 101 was in the state that is closest to the stationary state can be selected as the image to be recorded.

When the probe movement determiner 106 determines that the probe 101 was moving away from the surface of the subject's body at generation of a given ultrasound image from among the plurality of ultrasound images, the recording image selector 107 may select, from among the plurality of ultrasound images, an ultrasound image that was generated within a predetermined period that ends at the time the probe 101 was moved away from the surface of the subject's body. Here, the given ultrasound image may show that the probe 101 was stationary at the generation of the given ultrasound image. With this configuration, the operation of moving the probe 101 away from the body surface is regarded as a trigger of finishing the examination, and accordingly the image that the examiner would wish to record can be selected in response to the operation.

The probe movement determiner 106 may distinguish movement of the probe 101 from movement of tissues inside the subject's body, based on changes in each of the ultrasound images, the changes being indicated by the motion information of each of the ultrasound images. With this configuration, the movement in the entire ultrasound image and the movements in the image areas of the ultrasound image can be distinguished from each other, and the movement of the probe 101 can be determined independently from the movements of the inner tissues of the subject's body.

The image recording circuitry 151 may further include a probe movement state indicator 112 that indicates a probe movement state that is a result of determination performed by the probe movement determiner 106. Also, the image recording circuitry 151 may further includes a recording timing indicator 113 that indicates a recording timing of an ultrasound image selected by the recording image selector 107 from among the plurality of ultrasound images. With this configuration, the examiner is notified of the result of the probe movement determination, and the examiner can change the timing of the automatic recording.

The ultrasound diagnostic apparatus may be connectable to a display device 110, and may cause the display device 110 to display an ultrasound image selected by the recording image selector 107 from among the plurality of ultrasound images. This configuration allows for an easier freeze operation by which the image to be recorded, selected according to the probe movement determination, is continuously displayed on the display device 110.

The motion information may indicate a motion amount and a motion direction of each of a plurality of image areas of each of the plurality of ultrasound images. With respect to a given ultrasound image from among the plurality of ultrasound images, when the number of image areas whose motion amount is no greater than a reference value is no less than a predefined threshold value (i.e. the threshold value A), the probe movement determiner 106 may determine that the probe 101 was stationary at generation of the given ultrasound image, and when the number of the image areas is less than the predefined threshold value (i.e. the threshold value A), the probe movement determiner 106 may determine that the probe 101 was moving at the generation of the given ultrasound image. With this configuration, each ultrasound image is divided into a plurality of image areas, and the number of image areas belonging to any one of the ranges (i.e., bins) corresponding to the motion directions can be counted. As a result, the probe movement determination can be correctly performed based on each ultrasound image.

The motion direction detected from each of the plurality of image areas may belong to one of a plurality of motion angle ranges, and with respect to a given ultrasound image from among the plurality of ultrasound images, when the number of image areas whose motion amount is no greater than a reference value is less than a first threshold value (i.e. the threshold value A), the probe movement determiner 106 may determine that the probe 101 was stationary at the generation of the given ultrasound image when the number of image areas whose motion amount is greater than the reference value is less than a second threshold value (i.e. the threshold value B) with respect to every one of the plurality of motion angle ranges, the second threshold value (i.e. the threshold value B) being smaller than the first threshold value (i.e. the threshold value A), and the probe movement determiner 106 may determine that the probe 101 was moving at the generation of the given ultrasound image when the number of the image areas whose motion amount is greater than the reference value is no less than the second threshold value (i.e. the threshold value B) with respect to any one of the plurality of motion angle ranges. Here, the probe movement determiner 106 may determine that the probe 101 was stationary at generation of a given ultrasound image when the motion direction of each of the plurality of image areas of the given ultrasound image does not belong to a same one of the plurality of motion angle ranges. With this configuration, the movement of the entire ultrasound image and the movements of the image areas of the ultrasound image can be distinguished from each other. In other words, the movement of the probe 101 and the movements of the inner tissues of the subject's body can be distinguished from each other.

<Supplement>

The embodiment described above is merely one preferable example of the present invention. Values, forms, materials, components, component positions and connections, steps, step order, etc., illustrated in the embodiment represent examples and do not limit the spirit of the present invention. Further, among components of the embodiment, processes not disclosed in independent claims reciting top-level concepts of the present invention are described as components for further beneficial effect.

Further, to aid in understanding the invention, reduced scale components of each image of the embodiment may differ from actual implementation. Further, disclosure of the above embodiment is not a limitation and may be appropriately changed within the scope of the spirit of the present invention.

Furthermore, although materials such as circuit parts on a substrate, lead wires, etc., do exist in the ultrasound diagnostic device, electrical wiring and electric circuits may have a wide variety of implementations based on common knowledge in the technical field, and are therefore omitted from the description as they have no direct relevance to description of the present invention. Note that each drawing described above is schematic, and is not an exact representation.

Although the present invention has been fully described by way of examples with reference to the accompanying drawings, it is to be noted that various changes and modifications will be apparent to those skilled in the art. Therefore, unless such changes and modifications depart from the scope of the present invention, they should be construed as being included therein.

What is claimed is:

1. An ultrasound diagnostic apparatus to which a probe is connectable and that records an ultrasound image of the inside of a subject's body based on reflected ultrasound from inside the subject's body acquired by the probe positioned to face a surface of the subject's body, the ultrasound diagnostic apparatus comprising image recording circuitry that includes:
an ultrasound image generator that generates a plurality of ultrasound images of the inside of the subject's body based on the reflected ultrasound;
a probe movement determiner that determines, based on motion information obtained by analyzing the plurality of ultrasound images, movement of the probe occurring at generation of each of the plurality of ultrasound images;
a recording image selector that selects at least one ultrasound image from among the plurality of ultrasound images according to the determination performed by the probe movement determiner; and
an ultrasound image recorder that records therein said at least one ultrasound image selected by the recording image selector.

2. The ultrasound diagnostic apparatus of claim 1, wherein
in response to a determination by the probe movement determiner that the probe was stationary at generation of a given ultrasound image from among the plurality of ultrasound images,
the recording image selector selects the given ultrasound image.

3. The ultrasound diagnostic apparatus of claim 1, wherein
in response to a determination by the probe movement determiner that the probe was moving along the surface of the subject's body at generation of every one of the plurality of ultrasound images,
the recording image selector selects, from among the plurality of ultrasound images, an ultrasound image showing that the amount of movement of the probe was the smallest.

4. The ultrasound diagnostic apparatus of claim 1, wherein
in response to a determination by the probe movement determiner that the probe was moving away from the surface of the subject's body at generation of a given ultrasound image from among the plurality of ultrasound images,
the recording image selector selects, from among the plurality of ultrasound images, an ultrasound image that was generated within a predetermined period that ends at the time the probe was moved away from the surface of the subject's body.

5. The ultrasound diagnostic apparatus of claim 4, wherein
the given ultrasound image shows that the probe was stationary at the generation of the given ultrasound image.

6. The ultrasound diagnostic apparatus of claim 1, wherein
the probe movement determiner distinguishes movement of the probe from movement of tissues inside the subject's body, based on changes in each of the ultrasound images, the changes being indicated by the motion information of each of the ultrasound images.

7. The ultrasound diagnostic apparatus of claim 1, wherein
the image recording circuitry further includes a probe movement state indicator that indicates a probe movement state that is a result of determination performed by the probe movement determiner.

8. The ultrasound diagnostic apparatus of claim 1, wherein
the image recording circuitry further includes a recording timing indicator that indicates a recording timing of an ultrasound image selected by the recording image selector from among the plurality of ultrasound images.

9. The ultrasound diagnostic apparatus of claim 1, wherein
the ultrasound diagnostic apparatus is connectable to a display device, and causes the display device to display an ultrasound image selected by the recording image selector from among the plurality of ultrasound images.

10. The ultrasound diagnostic apparatus of claim 1, wherein
the motion information indicates a motion amount and a motion direction of each of a plurality of image areas of each of the plurality of ultrasound images.

11. The ultrasound diagnostic apparatus of claim 10, wherein
with respect to a given ultrasound image from among the plurality of ultrasound images, when the number of image areas whose motion amount is no greater than a reference value is no less than a predefined threshold value, the probe movement determiner determines that the probe was stationary at generation of the given ultrasound image, and when the number of the image areas is less than the predefined threshold value, the probe movement determiner determines that the probe was moving at the generation of the given ultrasound image.

12. The ultrasound diagnostic apparatus of claim 10, wherein
the motion direction detected from each of the plurality of image areas belongs to one of a plurality of motion angle ranges, and
with respect to a given ultrasound image from among the plurality of ultrasound images, when the number of image areas whose motion amount is no greater than a reference value is less than a first threshold value,
the probe movement determiner determines that the probe was stationary at the generation of the given ultrasound image when the number of image areas whose motion amount is greater than the reference value is less than a second threshold value with respect to every one of the plurality of motion angle ranges, the second threshold value being smaller than the first threshold value, and the probe movement determiner determines that the probe was moving at the generation of the given ultrasound image when the number of the image areas whose motion amount is greater than the reference value is no less than the second threshold value with respect to any one of the plurality of motion angle ranges.

13. The ultrasound diagnostic apparatus of claim 10, wherein
the motion angle detected from each of the plurality of image areas belongs to one of a plurality of motion angle ranges, and
the probe movement determiner determines that the probe was stationary at generation of a given ultrasound image when the motion direction of each of the plurality of image areas of the given ultrasound image does not belong to a same one of the plurality of motion angle ranges.

14. An ultrasound image recording method for recording an ultrasound image of the inside of a subject's body based on reflected ultrasound from inside the subject's body acquired by a probe positioned to face a surface of the subject's body, comprising:
generating a plurality of ultrasound images of the inside of the subject's body based on the reflected ultrasound;
determining, based on motion information of each of the plurality of ultrasound images obtained by analyzing the plurality of ultrasound images, movement of the probe occurring at generation of each of the plurality of ultrasound images;
selecting at least one ultrasound image from among the plurality of ultrasound images according to the determining; and
recording said at least one ultrasound image.

15. A non-transitory computer readable recording medium on which is recorded a program for causing a computer to perform the ultrasound image recording method of claim 14.
